(19) United States
(12) Patent Application Publication
(10) Pub. No.: US 2023/0242625 A1
(43) Pub. Date: Aug. 3, 2023

(54) PANDEMIC-PREPAREDNESS COCKTAIL TO FIGHT CORONAVIRUS OUTBREAKS

(71) Applicant: THE PENN STATE RESEARCH FOUNDATION, University Park, PA (US)

(72) Inventors: Deborah F. KELLY, Lemont, PA (US); Michael A. CASASANTA, State College, PA (US); Mariah L. SCHROEN, Lemont, PA (US); G.M. JONAID, State College, PA (US)

(21) Appl. No.: 18/009,895

(22) PCT Filed: Jun. 11, 2021
(86) PCT No.: PCT/US2021/037019

§ 371 Date: Dec. 12, 2022

Related U.S. Application Data
(60) Provisional application No. 63/038,286, filed on Jun. 12, 2020.
(60) Provisional application No. 63/151,470, filed on Feb. 19, 2021.

(51) Int. Cl. C07K 16/10; C07K 14/005; C12N 7/00; A61P 31/14

(52) U.S. Cl. CPC C07K 16/10; C07K 14/005; C12N 7/00; A61P 31/14; C12N 2770/20023

(57) ABSTRACT

Disclosed are novel targets for coronavirus therapies and methods of their use in screens for novel inhibitors of coronaviral infections. Also disclosed are novel coronavirus inhibitors that interact with said targets.

This application claims the benefit of U.S. Provisional Application No. 63/151,470, filed on Feb. 19, 2021, and U.S. Provisional Application No. 63/038,286, filed on Jun. 12, 2020, applications which are incorporated herein by reference in their entirety.

This invention was made with government support under Grant No. CA193578, CA227261, and CA219700 awarded by the National Institutes of Health. The government has certain rights in the invention.

### I. BACKGROUND

Starting in 2019 and continuing in 2020 a worldwide pandemic occurred from the coronavirus Severe Acute Respiratory Syndrome (SARS)-Coronavirus (CoV)-2 (SARS-CoV-2) causing the disease COVID-19 (coronavirus disease 2019). By June of 2020, over 7.5 million people had become infected and over 400,000 died from the infection. In the United States alone over 2 million people were infected and over 100,000 people died. To combat the virus, research institutes and pharmaceutical companies have been racing to develop therapeutics.

Coronavirus genome is fairly simple having just 4 proteins, viral spike glycoprotein (S) a viral envelope protein (E), and a membrane protein (M) which together form the viral envelope and the viral nucleocapsid. Most of the vaccine or therapeutics candidates focus attention of the viral spike glycoprotein (S), but to date no successful vaccine or therapeutic has been developed. What are needed are new vaccines and therapeutic agents that can treat or prevent viral infection and transmission.

### II. SUMMARY

Disclosed are methods and compositions related to novel targets for coronavirus therapies and methods of their use in screens for novel inhibitors of coronaviral infections.

In one aspect, disclosed herein are methods of screening for an anti-coronavirus therapeutic agent comprising contacting a coronavirus N protein with a therapeutic agent; wherein a therapeutic agent that binds to the N protein N-terminal “top hat” motif (residues 1-62 as set forth in SEQ ID NO: 1) or the N protein C-terminal helix (residues 362-419 as set forth in SEQ ID NO: 1) is an anti-coronavirus therapeutic agent. For example, the therapeutic agent can bind a B cell epitope including, but not limited to residues 42-62, residues, 153-172, or residues 355-401 as set forth in SEQ ID NO: 1 (such as, for example, the sequence motif X¹RQX²KQX³X⁴X⁵TLLPA including but not limited to SEQ ID NO: 3); and/or the therapeutic agent can bind a T cell epitope including, but not limited to residues 138-146, residues 159-167, residues 215-224, residues 219-227, residues 222-230, residues 226-234, residues 265-274, residues 316-324, residues 322-331, or residues 345-353 as set forth in SEQ ID NO: 1.

Also disclosed herein are methods of screening for an anti-coronavirus therapeutic agent of any preceding aspect, wherein the interaction of the therapeutic agent and the N protein is measured by mass spectrometry, crystallography, neutron diffraction, proteolysis, nuclear magnetic resonance (NMR), electron paramagnetic resonance (EPR), cryogenic electron microscopy (cryoEM), laser spectroscopy, electron crystallography, enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP)

In one aspect, disclosed herein are anti-coronavirus therapeutic agents identified by the method of screening for an anti-coronavirus therapeutic agent of any preceding aspect. For example, disclosed herein are anti-coronavirus therapeutic agents (such as, for example, a small molecule, antibody, antibody fragment, RNAi, siRNA, peptide, or protein, or any combination thereof); wherein the anti-coronavirus therapeutic agent binds to the N-terminal “top hat” motif (residues 1-62) or the C-terminal helix (residues 362-419) of the nucleocapsid (N) protein of a coronavirus, including, but not limited to therapeutic agents can bind a B cell epitope including, but not limited to residues 42-62, residues 153-172, or residues 355-401 as set forth in SEQ ID NO: 1 (such as, for example, the sequence motif X¹RQX²KQX³X⁴X⁵TLLPA including but not limited to SEQ ID NO: 3); and/or the therapeutic agent can bind a T cell epitope including, but not limited to residues 138-146, residues 159-167, residues 215-224, residues 219-227, residues 222-230, residues 226-234, residues 265-274, residues 316-324, residues 322-331, or residues 345-353 as set forth in SEQ ID NO: 1.

Also disclosed herein are recombinant viruses or virus-like particles comprising a nucleic acid encoding the N protein of a coronavirus (including, but not limited to avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), and middle east respiratory syndrome (MERS) coronavirus (CoV) (MERS-CoV)) or a fragment thereof (such as, for example, the N-terminal “top hat” motif of the N-protein and/or the C-terminal helix of the N-protein). For example, disclosed herein are recombinant viruses or virus-like particles of any preceding aspect, wherein the recombinant virus or VLP comprises a nucleic acid encoding the sequence motif X¹RQX²KQX³X⁴X⁵TLLPA; wherein X¹ is Q or K; wherein X² is K or R; wherein X³ is P. Q, or G; wherein X⁴ is T or S; and wherein X⁵ is V or I (such as, for example SEQ ID NO: 3); or wherein the recombinant virus or VLP comprises residues 138-146, residues 159-167, residues 215-224, residues 219-227, residues 222-230, residues 226-234, residues 265-274, residues 316-324, residues 322-331, or residues 345-353 as set forth in SEQ ID NO: 1.

In one aspect, disclosed herein are immunogenic compositions comprising one or any combination of two or more of a N-terminal “top hat” motif or the C-terminal helix of the nucleocapsid (N) protein of a coronavirus of any preceding aspect, a recombinant virus or virus-like particle comprising a nucleic acid encoding the N protein of a coronavirus or a fragment thereof of any preceding aspect, or an antibody or antibody fragment to the N-terminal “top hat” motif or the C-terminal helix of the N protein of a coronavirus.

Also disclosed herein are methods of treating, inhibiting, decreasing, reducing, ameliorating, and/or preventing a coronavirus infection (such as, for example, avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), and middle east respiratory syndrome (MERS) coronavirus (CoV) (MERS-CoV)) in a subject comprising administering to the subject the anti-coronavirus therapeutic agent, the immunogenic composition, the recombinant virus, and/or the virus-like particle, of any preceding aspect.

In one aspect, disclosed herein are methods of preventing or inhibiting a Severe Acute Respiratory Syndrome (SARS)-Coronavirus (CoV)-2 (SARS-CoV-2) infection in a subject comprising immunizing the subject with human coronavirus (HCoV)-229E, HCoV-OC43, HCoV-HKU1, HCoV-NL63 or a recombinant virus or virus-like particle comprising the N protein of a coronavirus or a fragment thereof (such as, for example, the N-terminal “top hat” motif of the N-protein and/or the C-terminal helix of the N-protein). In one aspect, the N protein or a fragment thereof is derived from the N-protein of a coronavirus selected from the group consisting of avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), and middle east respiratory syndrome (MERS) coronavirus (CoV) (MERS-CoV).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A and 1B show that antibody results agree with viral PCR results within ˜30 days of testing. FIG. 1A shows that test kits contain recombinant N protein substrate from SARS-CoV-2 and are commercially available from RayBiotech, Inc. A band at the control “C” region indicates a valid test. A positive band at the test region “T” (left) indicates the presence of IgG antibodies in test samples. The absence of a band in the test region (right) indicates no IgG antibodies were detected. FIG. 1B shows that data are in good agreement between individuals that were IgG+ or IgG− within 30 days of PCR test results.

FIGS. 2A, 2B, and 2C show analysis of common cold-like symptoms and coronavirus proteins. FIG. 2A shows that individuals who were medically diagnosed with COVID-19 did not have prior CC symptoms (IgG+/CC−) in months prior to the outbreak (November 2019-February 2020). Many individuals did not have detectable levels of antibodies but did have CC-like symptoms (IgG−/CC+) in months prior to the COVID-19 pandemic. Five individuals did not report CC symptoms nor were antibodies noted (IgG−/CC−). In shared dwellings with IgG+ individuals, those with prior CC symptoms were IgG−. No individuals were found to be IgG+ that had prior CC symptoms. FIG. 2B shows the percent sequence identity among common cold coronaviruses N proteins in comparison the SARS-CoV-2 N protein. FIG. 2C shows a Cryo-EM image of the SARS-CoV-2 N protein. Scale bar is 20 nm.

FIG. 3 shows structural comparisons of N protein models derived from human pathogens. Cryo-EM structure (left, red) of the SARS-CoV-2 coronavirus N protein determined in the Kelly Lab at Penn State University. The EM density map is interpreted using a model from consensus structures of individual protein domains. Additional models were calculated using the PHYRE2 protein folding server and the model template. The N-terminal motif and C-terminal helix are unique to SARS-CoV-2 in comparison to other N proteins from human pandemic and common cold strains. Percent sequence identity is indicated according to BLAST sequence alignment tools.

FIG. 4 shows structural comparisons of N protein models derived from “wild life” species and human pathogens. Models were calculated using the PHYRE2 protein folding server in comparison to the SARS-CoV-2 structure (red, far right). The N-terminal motif that is unique in SARS-CoV-2 is also found in SARS-CoV N protein from civets (orange, right, 89% sequence identity). The Bat N protein model is highly similar to SARS-CoV from humans but shares ˜90% sequence identity with human SARS-CoV-2 N protein according to BLAST sequence alignment tools.

FIG. 5 shows the use of functionalized microchips to prepare low-molecular weight proteins for cryo-EM. (Step 1) The SARS-CoV-2 N protein in solution was validated by SDS-PAGE and Native gel analysis. N protein migrates at 50 kDa according to SimplyBlue-stained gels and western blots probed against the His-tag (IB: immunoblot). (Step 2) Silicon nitride microchips (2 mm×2 mm frames) were coated with Ni-NTA layers (yellow), spread over an array of microwells (10 μm×10 μm each). Etched imaging windows were 20-nm thin and the depth of each microwell was 150 nm. Microchip samples were vitrified in liquid ethane and maintained at −180° C. until examined in the TEM. (Step 3) Specimens can be imaged using a variety of high-resolution instruments such as the Talos F200X, Talos F200C, or Titan TEM/STEM.

FIGS. 6A, 6B, 6C, and 6D show quality assessment of single particle data for N protein specimens. FIG. 6A shows images and class averages of frozen-hydrated N protein particles show consistent features from multiple views. Scale bar is 20 nm, box size is 10 nm. FIG. 6B shows the angular distribution plot of particle orientations lacks major limitations. FIG. 6C shows the Fourier shell correlation (FSC) curve and Cref (0.5) evaluation indicate a spatial resolution of 4.5-Å at the 0.143 value using the gold-standard (GS) criteria. FIG. 6D shows the calculated density map of the N protein at 4.5-Å resolution (yellow) is in good agreement with the experimental EM map (gray), scale bar is 10 Å.

FIGS. 7A, 7B, and 7C show microchip-enabled cryo-EM structure of the SARS-CoV-2 N protein. FIG. 7A shows cryo-EM map resolved to 4.5-Å shows distinctive N- and C-terminal domains with a unique “top hat” motif in the first 50-amino acids of the protein. The map was interpreted with a model for the SARS-CoV-2 N protein (red) calculated using consensus structures in the PHYRE2 server. Rotational views along with a magnified section of the map provide detailed information of the flexible (N-terminal) and rigid (C-terminal) features of the structure. The central helix in the structure from residues D216 to N228 defined a boundary between the two domains. Scale bar is 10 Å. FIG. 7B shows surface rendering of the N protein in different views show patches of basic residues in the N-terminus. The C-terminal region contains 3D pockets and clefts for substrates or binding partners. Predicted epitopes mapped onto the N protein surface were evaluated according to their accessibility as high, limited or buried. Highly accessible residues are noted. FIG. 7C shows for nucleotide binding assays, N-protein samples were incubated with SARS-CoV-2/PCR+ serum in PBS at 37° C. for 60 minutes. Reaction mixtures were halted with sample gel loading buffer containing no SDS. Samples were assessed using native gels and immunoblots. N protein migrated at 50 kDa in mixtures lacking viral RNA (RNA−). Control reactions lacking N protein did not show background proteins. Mixtures containing N protein and PCR+ serum (+N/+RNA) showed a shift in the N protein band to a higher molecular weight. Western blots were probed with primary antibodies against the His tag on the N protein.

FIG. 8 shows molecular dynamics (MD) simulations show flexible loop domains in the N-terminal top hat region. The N-terminal residues of the N protein (Met¹-Thr⁴⁹) were examined using MD simulations integrated into the Chimera Software package. Minimization parameters included 100 steepest descent steps with a step size of 0.02 Å, along with 10 conjugate gradient steps with a step size of 0.02 Å. Charges were assigned for minimization purposes using the AMBER force field (AMBER FF14SB). Simulations were performed on the initial structure for up to 300 frames of movie output. Met¹ (black arrows) was either (1) held in place to represent the his-tagged “tethered” construct (top panel), or (2) allowed to move freely representing the “untethered” protein (bottom panel). Changes in the wire frame rendering of the protein segment show comparable differences in dynamic movements.

FIGS. 9A and 9B show a comparison of microchip-tethered and untethered N protein structures. FIG. 9A shows EM structure of the his-tagged N protein sample that was tethered to the microchip substrate (purple) and resolved at 4.5-Å. FIG. 9B shows an untethered structure of the N protein (blue) interacted with microchip substrates through putative electrostatic charges (similar to glow-dicharged EM grids). The EM structures showed good agreement. Angular distribution plots of particle orientations (A, B) lacked major limitations and show a potentially greater number of particle views in the untethered structure. Scale bar is 10 Å.

FIGS. 10A, 10B, 10C, 10D, and 10E show predicted and experimental evidence for N protein-antibody interactions. FIG. 10A shows N protein samples were incubated with the Ni-NTA coated microchips, followed by serum containing IgG antibodies. Chip contents were assessed using SDS-PAGE analysis. N protein migrated at 50 kDa and was the only band present in −IgG controls. Samples with N protein and antibodies (+N/+IgG) showed bands for the IgG heavy chain (HC), light chain (LC), and the N protein. Controls lacked N protein (−N/+IgG). A separate purified IgG control sample demonstrates the manner in which IgG antibodies migrate on a denaturing gel. FIG. 10B shows that antibody test cassettes contain the His-tagged N protein. Within 10 minutes after applying the reaction mixture serum, a band at the control “C” region indicates a valid test. A positive band at the test region “T” (black arrow, left) indicates the presence of IgG antibodies abound to the N protein analyte. The absence of a band in the test region (right) indicates no detectable IgGs. FIG. 10C shows an image of the N protein incubated with patient antibodies. Class averages of antibody-bound N protein (+ Abs) show density (red arrows) not present in controls (−Abs). FIG. 10D shows particle orientations shows sufficient views without major limitations angular. FIG. 10E shows the GS-FSC curve and Cref(0.5) evaluation indicate 14.2-Å resolution.

FIGS. 11A and 11B show Cryo-EM structure of N protein decorated with a Fab fragment from COVID-19 serum. FIG. 11A shows Cryo-EM map resolved to 14.2-Å shows the placement of the N protein (red) and a corresponding model for a Fab fragment (cyan). The model for the N protein fits in one orientation within the map with the C-terminal domain adjacent to the antigen-binding domain in the Fab model. The flexible loop comprised of residues Q384-A397 is proximal to the Fab-binding site. Rotational views of the map provide visual clarity of the physical relationship between the two models. Scale bar is 10 Å. FIG. 11B shows cross-sections through the reconstruction indicate a high-quality fit of the models from side and top views. Sections through the map represent slices produced at ˜10-Å increments.

FIGS. 12A and 12B show structural comparisons of N protein models. FIG. 12A shows that N protein models were calculated using the PHYRE2 server and multiple consensus templates with the highest structural correlational values. The N- and C-terminal domains are unique to SARS-CoV-2 in comparison to other N proteins from human pandemic and common cold strains. Percent sequence identities were generated according to multi-sequence alignment tools and included: Civet, 89.5%; SARS-CoV, 89.7%; MERS 48.6%, OC43, 35.9% HKU1, 35.7%, NL63, 27.9%, and 229E, 25.2%. Proteins were aligned visually to highlight similarities and differences among the predicted structures. Structures are oriented with the N-terminus at the vertical top, the C-terminus at the vertical bottom, and putative antibody epitopes on the right. FIG. 12B shows that the structural dendrogram indicates similarities between N protein models, demonstrated in the specified branches and groupings by the DALI protein server. Alpha-coronaviruses (229E and NL63) appear on similar branches and in long range opposition to beta-coronaviruses (OC43 and HKU1). Pandemic beta-coronaviruses, SARS-CoV and SARS-CoV-2, are located in the central branch. This proximity suggests a mixture of features are represented in the structures.

FIG. 13. Multi-sequence alignment results for pandemic and CC coronavirus N proteins. Primary amino acid sequences for each N protein are listed in comparison to the SARS-CoV-2 pandemic strain. Sequences were obtained from ViPR and uploaded to the ClustalOmega online software package to generate percent identity matrices. Output shows identical amino acids in red and those with similar side chain properties are in blue.

FIG. 14. Multi-sequence alignment results for CC coronavirus N proteins. Primary amino acid sequences for each CC N protein were obtained from ViPR and are shown in comparison to 229E coronavirus strain. Sequences were uploaded to the ClustalOmega online software package to generate percent identity matrices. Output is displayed with identical amino acids in red and those with similar side chain properties colored in blue.

FIG. 15. Multi-sequence alignment results for predicted epitope among pandemic strains. Primary amino acid sequences for the potential antibody binding site among pandemic strains are aligned for easy visual comparison. Percent identities were calculated using ClustalOmega and are recorded in the corresponding table. Output is displayed with identical amino acids in red and those with similar side chain properties colored in blue.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The term includes small molecule compounds, antisense reagents, siRNA reagents, RNAi reagents, antibodies, diabodies, immunotoxins, enzymes, peptides organic or inorganic molecules, cells, natural or synthetic compounds and the like. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular small molecule, RNAi, antibody, peptide, or protein that binds to the nucleocapsid protein (N) of SARS-CoV-2 is disclosed and discussed and a number of modifications that can be made to a number of molecules including the small molecule, RNAi, antibody, peptide, or protein that binds to the nucleocapsid protein (N) of SARS-CoV-2 are discussed, specifically contemplated is each and every combination and permutation of small molecule, RNAi, antibody, peptide, or protein that binds to the nucleocapsid protein (N) of SARS-CoV-2 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

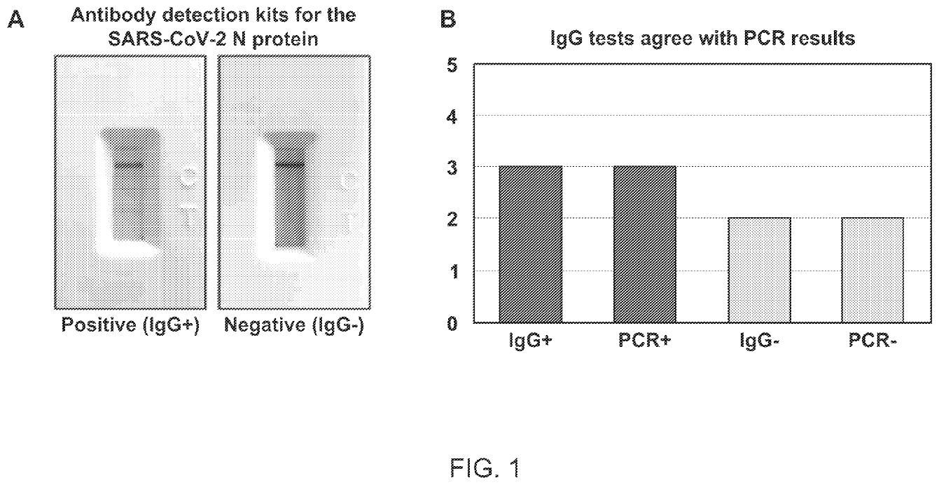

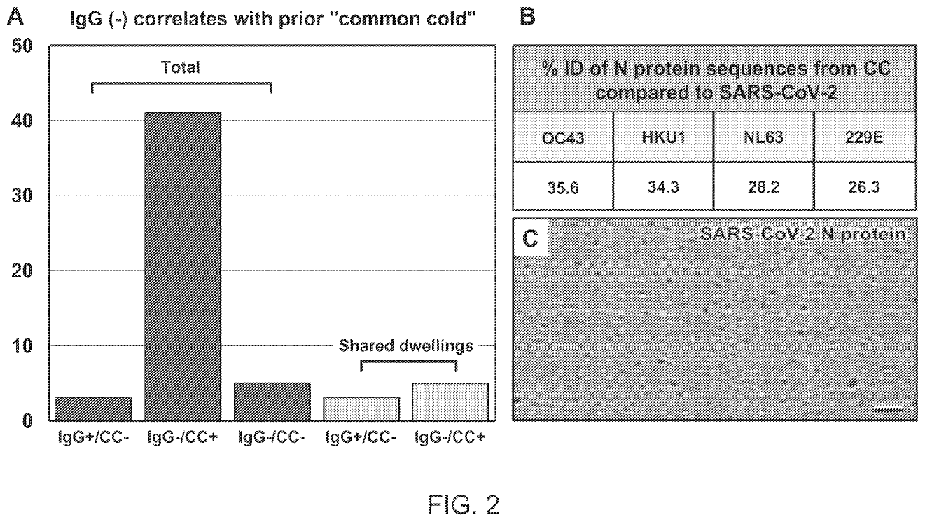

Data provided from PCR-test results and correlative antibody (IgG) information (FIG. 1) were examined for individuals in different parts of the United States from March-May 2020, in the middle of the COVID-19 pandemic. Individuals having prior CC-like symptoms in the months leading up to the pandemic (November 2019-February 2020) were negative for IgGs against the SARS-CoV-2 N Protein (FIG. 2A). These people did not seek hospitalization for any recent symptoms. Individuals with PCR+/IgG+ results had no CC-like symptoms in the months leading up to the pandemic. This finding was consistent for IgG− individuals who lived in shared dwellings with PCR+/IgG+ individuals. IgG− individuals in shared dwellings with PCR+/IgG+ individuals all reported CC-like symptoms in months prior to the pandemic. Taken together, these observations indicated that a recent potential exposure to a human coronavirus associated with the common cold can provide some degree of immune cross-reactivity against SARS-CoV-2.

Most vaccine candidates and therapeutics under development for SARS-CoV-2 are designed against some portion of the viral S protein. Herein is disclosed the utilization of unique features that were discovered in the Nucleocapsid (N) protein that is found internally within the virus capsid. The N protein is the most abundant protein in pathogenic coronaviruses and it is presumed to be the most antigenic. This information is based on the substantive fact that most antibody testing kits are designed to recognize antibodies against the N protein from blood or plasma. Drugs or antibody therapies used to neutralize the N protein are not being heavily investigated at the moment and we have discovered the first 3D structural insights of the N protein from SARS-CoV-2.

To search for similarities between CC-related coronaviruses and SARS-CoV-2, primary sequences of the N protein from OC43, HKU1, NL63, and 229E CC-strains were compared (FIG. 2B). The percent identities in primary sequences were moderate in comparison to SARS-CoV-2. Unfortunately, there is little structural information available for each of these proteins. Determining the 3D structure for the SARS-CoV-2, however, presented molecular modeling opportunities for further comparisons. Cryo-EM specimens were prepared from commercially available recombinant SARS-CoV-2 N protein expressed and purified from bacteria (FIG. 2C). Standard data collection was performed on the FEI Talos F200C electron microscope at Penn State operating at 200 kV.

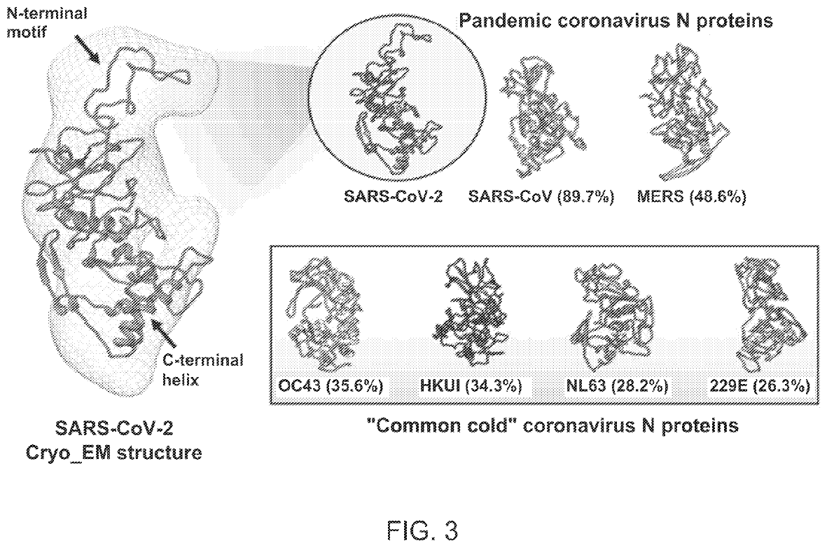

The initial density map (FIG. 3, left panel) revealed a unique and distinct protein fold in the first approximately 50-amino acid residues of the N protein, referred to as a “top hat” motif. This feature cannot be predicted solely from amino acid sequence analysis. This region can interact with nucleic acids, such as viral RNA or host nucleotides. The N protein fold extends from residues 1-156. In addition, the C-terminal ˜50 amino acids (residues 362-419) of the SARS-CoV-2 N protein form a rigid helix domain that forms epitopes for antibody interactions. For the C-terminus, the target helix residues are 400-419. This information was used for homology modeling analysis for other pandemic-related N proteins for SARS (89.7% ID) and MERS (48.6% ID) (FIG. 3, right panel). By comparison, the unique N-terminal motif and C-terminal features in the SARS-CoV-2 protein to be suitable targets for drug discovery or antibody treatment. Based on these structural insights along with other crystallographic information, reliable models for the common cold-related N proteins were produced (FIG. 3, bottom panel).

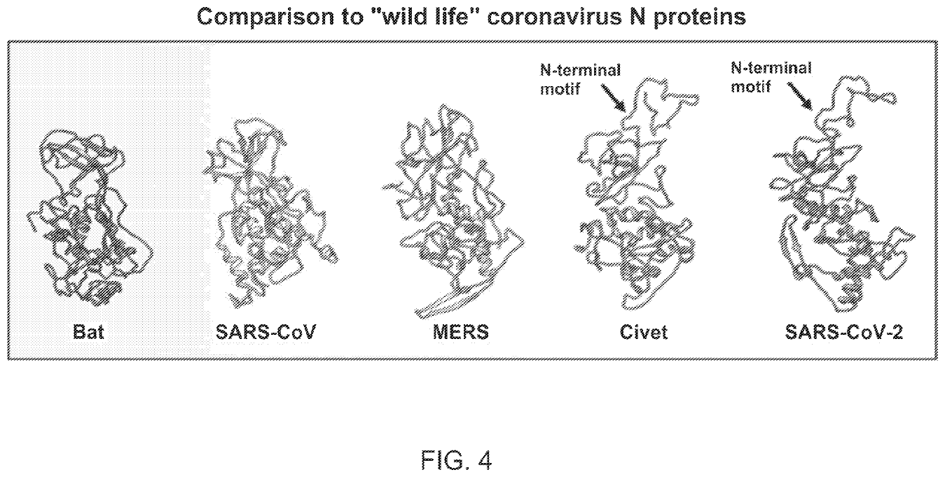

Structural conservation is apparent in the putative nucleic acid binding region located in the N-terminus of these proteins, although the extended “top hat” feature of the SARSCoV-2 N-terminus remains unique. While there is structural variability in the C-terminal domain of each protein, this region provides antigenic similarities among antibody targets. Similar protein folds in the C-terminal region are a source of cross-reactivity among IgG antibodies in test samples. Additional insights to improve viral derivatives can be sought from N-proteins sources found in different wild life sources as shown in FIG. 4.

The findings suggest that N proteins derived from SARS-CoV-2 or other “common cold” strains of human coronaviruses can stimulate the immune system by producing cross-reactive antibodies against COVID-19 culprits.

Accordingly, in one aspect, disclosed herein are methods of screening for an anti-coronavirus therapeutic agent comprising contacting a coronavirus N protein with a therapeutic agent; wherein a therapeutic agent that binds to the N protein N-terminal “top hat” motif or the N protein C-terminal helix is an anti-coronavirus therapeutic agent.

Also disclosed herein are methods of screening for an anti-coronavirus therapeutic agent of any preceding aspect, wherein the interaction of the therapeutic agent and the N protein is measured by mass spectrometry, crystallography, neutron diffraction, proteolysis, nuclear magnetic resonance (NMR), electron paramagnetic resonance (EPR), cryogenic electron microscopy (cryoEM), laser spectroscopy, electron crystallography, enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

1. Compositions Identified by Screening with Disclosed Compositions/

As noted above, the disclosed N-terminal “top hat” motif and C-terminal helix of the coronavirus (including, but not limited to avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), and middle east respiratory syndrome (MERS) coronavirus (CoV) (MERS-CoV) N protein can be used as targets for any combinatorial technique, imaging technique, and/or immunological technique (including, but not limited to mass spectrometry, crystallography, neutron diffraction, proteolysis, nuclear magnetic resonance (NMR), electron paramagnetic resonance (EPR), cryogenic electron microscopy (cryoEM), laser spectroscopy, electron crystallography, enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP)) to identify molecules or macromolecular molecules that interact with the disclosed N-terminal “top hat” motif and C-terminal helix of the coronavirus N protein in a desired way. These structural motifs of the N protein disclosed herein can be used as targets for the combinatorial approaches. Also disclosed are the compositions that are identified through combinatorial techniques or screening techniques in which the N-terminal “top hat” motif and C-terminal helix of the coronavirus N protein are used as the target in a combinatorial, immunological, and/or imaging screening protocol.

It is understood that the disclosed methods for identifying molecules that inhibit the interactions between, for example, an antibody and the N-terminal motif and C-terminal helix of the coronavirus N protein can be performed using cryoEM as well as high through put means. For example, putative inhibitors can be identified using Fluorescence Resonance Energy Transfer (FRET) to quickly identify interactions.

CryoEM can be used to directly visualize and identify therapeutic agents bound to N protein structures. Computational methods transform the images collected from cryoEM data into 3D density maps. Extra components found in the resulting density maps that are not a part of the N protein represent the bound therapeutic agents.

The underlying theory of the techniques is that when two molecules are close in space, ie, interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Given the identification of targets for therapies directed against coronaviral infections, it is understood and herein contemplated that the screens disclosed herein are designed to result in the identification of novel therapeutic agents. Thus, disclosed herein are anti-coronavirus therapeutic agents identified by the method of screening for an anti-coronavirus therapeutic agent. As disclosed herein, the therapeutic agent can be a small molecule, antibody, antibody fragment, RNAi, siRNA, peptide, or protein, or any combination thereof that binds to the N-terminal “top hat” motif or the C-terminal helix of the nucleocapsid (N) protein of a coronavirus.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2, 3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

a) Computer Assisted Drug Design

The disclosed compositions can be used as targets for any molecular modeling technique to identify either the structure of the disclosed compositions or to identify potential or actual molecules, such as small molecules, which interact in a desired way with the disclosed the N-terminal motif and C-terminal helix of the coronavirus N protein. Thus, the N-terminal motif and C-terminal helix of the coronavirus N-terminal “top hat” motif and/or C-terminal helix of the N protein of a coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV) N protein disclosed herein can be used as targets in any molecular modeling program or approach.

It is understood that when using the disclosed the N-terminal “top hat” motif and C-terminal helix of the coronavirus N protein in modeling techniques, molecules, such as macromolecular molecules, will be identified that have particular desired properties such as inhibition or stimulation or the target molecule's function. The molecules identified and isolated when using the disclosed compositions are also disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

2. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the N-terminal “top hat” motif and/or C-terminal helix of the N protein of a coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV). The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain N-terminal “top hat” motif and/or C-terminal helix of the N protein of a coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV) binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to 15 immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti N-terminal “top hat” motif and/or C-terminal helix of the N protein of a coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2 (including, but not limited to the B1.351 variant, B.1.1.7 variant, and P.1 variant), or MERS-CoV) antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

3. Delivery of the Compositions to Cells

There are a number of compositions and methods which can be used to deliver proteins (including, but not limited to the N-protein of a coronavirus (such as, for example SARS-CoV-2), peptides, fragment thereof (including fragments comprising the N-terminal “top hat” motif or a coronavirus N protein and/or C-terminal helix of a coronavirus N protein) and/or nucleic acids encoding said proteins and peptides to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as a nucleic acid encoding the N-protein of a coronavirus or a fragment thereof into the cell without degradation and include a promoter yielding expression of the gene in the cells into which it is delivered. Thus, in one aspect, disclosed herein are recombinant viruses or virus-like particles (VLPs) comprising nucleic acid encoding the N protein of a coronavirus (including, but not limited to avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), and middle east respiratory syndrome (MERS) coronavirus (CoV) (MERS-CoV)) or a fragment thereof (such as, for example, the N-terminal “top hat” motif of the N-protein and/or the C-terminal helix of the N-protein). For example, disclosed herein are recombinant viruses or virus-like particles (VLPs), wherein the recombinant virus or VLP comprises a nucleic acid encoding the sequence motif X¹RQX²KQX³X⁴X⁵TLLPA; wherein X¹ is Q or K; wherein X² is K or R; wherein X³ is P. Q, or G; wherein X⁴ is T or S; and wherein X⁵ is V or I (such as, for example SEQ ID NO: 3); or wherein the recombinant virus or VLP comprises residues 138-146, residues 159-167, residues 215-224, residues 219-227, residues 222-230, residues 226-234, residues 265-274, residues 316-324, residues 322-331, or residues 345-353 as set forth in SEQ ID NO: 1

Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J Neuroscience 5:1287-1291 (1993); and Ragot, J Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorproated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed antibodies, antibody fragments, small molecules, proteins, peptides, siRNA, RNAi, VLPs, recombinant viruses, or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other speciifc cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes ß-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

5. Immunogenic Compositions

In one aspect, disclosed herein are immunogenic compositions comprising one or any combination of two or more of a N-terminal “top hat” motif or the C-terminal helix of the nucleocapsid (N) protein of a coronavirus or a peptide fragment thereof, virus comprising the N protein of a coronavirus or a fragment thereof, virus like particle, or an antibody or antibody fragment to the N-terminal “top hat” motif or the C-terminal helix of the N protein of a coronavirus. For example, disclosed herein are immunogenic compositions comprising the sequence motif X¹RQX²KQX³X⁴X⁵TLLPA; wherein X¹ is Q or K; wherein X² is K or R; wherein X³ is P. Q, or G; wherein X⁴ is T or S; and wherein X⁵ is V or I (such as, for example, residues 384-397 of the SAR-CoV-2 N protein as set forth in SEQ ID NO 3); or immunogenic compositions comprising residues 138-146, residues 159-167, residues 215-224, residues 219-227, residues 222-230, residues 226-234, residues 265-274, residues 316-324, residues 322-331, or residues 345-353 as set forth in SEQ ID NO: 1.

6. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHOD OF TREATMENT

It is understood and herein contemplated that the therapeutic agents identified the disclosed screening methods as well as the proteins, peptides, recombinant viruses, VLP, and immunogenic compositions disclosed herein can be used to treating, inhibiting, reducing, ameliorating, and/or preventing a coronavirus infection or induce an immune response to a coronavirus infection. Accordingly, disclosed herein are methods of treating, inhibiting, decreasing, reducing, ameliorating, and/or preventing a coronavirus infection (such as, for example, avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), and middle east respiratory syndrome (MERS) coronavirus (CoV) (MERS-CoV)) in a subject comprising administering to the subject any of the anti-coronavirus therapeutic agents, immunogenic compositions, recombinant viruses, and/or the virus-like particles disclosed herein. For example, disclosed herein are methods of treating, inhibiting, decreasing, reducing, ameliorating, and/or preventing a coronavirus infection in a subject comprising administering to the subject an anti-coronavirus therapeutic agents (such as, for example, a small molecule, antibody, antibody fragment, RNAi, siRNA, peptide, or protein, or any combination thereof); wherein the anti-coronavirus therapeutic agent binds to the N-terminal “top hat” motif (residues 1-62) or the C-terminal helix (residues 362-419) of the nucleocapsid (N) protein of a coronavirus, including, but not limited to therapeutic agents can bind a B cell epitope including, but not limited to residues 42-62, residues 153-172, or residues 355-401 as set forth in SEQ ID NO: 1 (such as, for example, the sequence motif X¹RQX²KQX³X⁴X⁵TLLPA including but not limited to SEQ ID NO: 3); and/or the therapeutic agent can bind a T cell epitope including, but not limited to residues 138-146, residues 159-167, residues 215-224, residues 219-227, residues 222-230, residues 226-234, residues 265-274, residues 316-324, residues 322-331, or residues 345-353 as set forth in SEQ ID NO: 1. Also, for example, disclosed herein are methods of treating, inhibiting, decreasing, reducing, ameliorating, and/or preventing a coronavirus infection in a subject comprising administering to the subject an immunogenic composition comprising the sequence motif X¹RQX²KQX³X⁴X⁵TLLPA; wherein X¹ is Q or K; wherein X² is K or R; wherein X³ is P. Q, or G; wherein X⁴ is T or S; and wherein X⁵ is V or I (such as, for example, residues 384-397 of the SAR-CoV-2 N protein as set forth in SEQ ID NO 3); or immunogenic compositions comprising residues 138-146, residues 159-167, residues 215-224, residues 219-227, residues 222-230, residues 226-234, residues 265-274, residues 316-324, residues 322-331, or residues 345-353 as set forth in SEQ ID NO: 1.

Alternatively or in addition, disclosed herein are methods of treating, inhibiting, decreasing, reducing, ameliorating, and/or preventing a coronavirus infection in a subject comprising administering to the subject a recombinant virus or virus-like particle comprising nucleic acid encoding the N protein of a coronavirus (including, but not limited to avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), and middle east respiratory syndrome (MERS) coronavirus (CoV) (MERS-CoV)) or a fragment thereof (such as, for example, the N-terminal “top hat” motif of the N-protein and/or the C-terminal helix of the N-protein). For example, disclosed herein are recombinant viruses or virus-like particles, wherein the recombinant virus or VLP comprises nucleic acid encoding the sequence motif X¹RQX²KQX³X⁴X⁵TLLPA; wherein X¹ is Q or K; wherein X² is K or R; wherein X³ is P. Q, or G; wherein X⁴ is T or S; and wherein X⁵ is V or I (such as, for example SEQ ID NO: 3); or wherein the recombinant virus or VLP comprises nucleic acids encoding residues 138-146, residues 159-167, residues 215-224, residues 219-227, residues 222-230, residues 226-234, residues 265-274, residues 316-324, residues 322-331, or residues 345-353 as set forth in SEQ ID NO: 1.

In one aspect, disclosed herein are methods of preventing or inhibiting a Severe Acute Respiratory Syndrome (SARS)-Coronavirus (CoV)-2 (SARS-CoV-2) infection in a subject comprising immunizing the subject with human coronavirus (HCoV)-229E, HCoV-OC43, HCoV-HKU1, HCoV-NL63; protein, peptide, nucleic acid (DNA or RNA), or a recombinant virus or virus-like particle comprising the N protein of a coronavirus or a fragment thereof (such as, for example, the N-terminal “top hat” motif of the N-protein and/or the C-terminal helix of the N-protein including, but not limited to N protein fragments comprising the sequence motif X¹RQX²KQX³X⁴X⁵TLLPA; wherein X¹ is Q or K; wherein X² is K or R; wherein X³ is P. Q, or G; wherein X⁴ is T or S; and wherein X⁵ is V or I (such as, for example, such as, for example, residues 384-397 of the SAR-CoV-2 N protein as set forth in SEQ ID NO 3); and/or wherein the N-protein fragment comprises residues 138-146, residues 159-167, residues 215-224, residues 219-227, residues 222-230, residues 226-234, residues 265-274, residues 316-324, residues 322-331, or residues 345-353 as set forth in SEQ ID NO: 1. In one aspect, the N protein or a fragment thereof is derived from the N-protein of a coronavirus selected from the group consisting of avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), and middle east respiratory syndrome (MERS) coronavirus (CoV) (MERS-CoV).

### D. EXAMPLES

### 1. Example 1: Microchip-Based Structure Determination of Low-Molecular Weight Proteins Using Cryo-Electron Microscopy

a) Results

(1) Specimen Preparation and Validation of the N Protein.

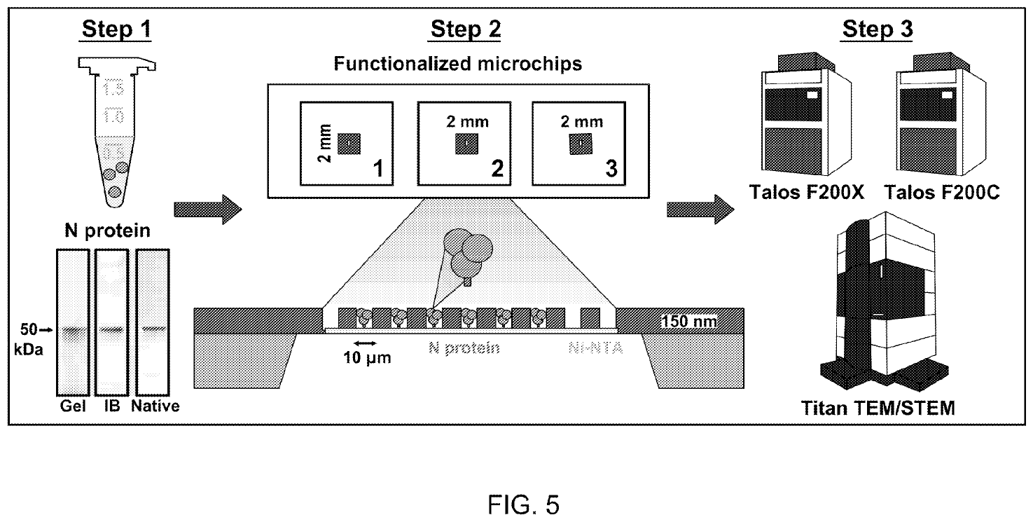

The SARS-CoV-2 N protein was expressed and purified from bacteria and is available from RayBiotech, Inc. along with other suppliers (RayBiotech, Cat. #230-01104-100). Biochemical validation of the N protein revealed a highly purified sample that migrated at 50 kDa according to SDS-PAGE analysis. Western blots probed with primary antibodies against the N-terminal His tag of the protein showed a single band at 50 kDa (FIG. 5, step 1). Other recent reports have shown multimerization of N protein from related viruses is possible. To confirm the predominance of N protein monomers in the sample, we performed Native-PAGE analysis which also showed a single band appearing at 50 kDa. The monomer status of the N protein is likely the result of the mild, physiologically relevant buffering conditions used in the experiments.

To prepare cryo-EM specimens, we employed microwell-integrated microchips (Protochips, Inc., EPB-42 Å1-10) with imaging windows having dimensions of 10 μm×10 μm in the x- and y-dimension and ˜20-nm thick. The etched windows are transparent in the beam of an electron microscope. Microchips were cleaned by submerging in acetone for 2 minutes, followed by methanol for 2 minutes. Cleaned microchips were coated with 25% Ni-NTA-containing lipid monolayers. Aliquots (2 μL) of His-tagged N protein (0.1 mg/mL in 20 mM Tris (pH 7.5), 150 mM NaCl, 10 mM MgCl₂, 10 mM CaCl₂)) were added to the Ni-NTA-coated microchips and incubated for 1 minute at room temperature (FIG. 5, step 2).

The electrostatic properties of proteins are based on their charge distributions, which are heavily influenced by local pH conditions. Preparing cryo-EM samples of proteins in biologically relevant conditions is likely to yield more accurate information about its true structural state, rather than subjecting samples to extreme salt concentrations or non-native additives. Microchip samples prepared under mild buffering conditions were loaded into a FEI Mark III Vitrobot and flash-frozen into liquid ethane. Specimens were placed in the tip of a 626 Gatan specimen holder and transferred to a Talos F200C TEM (ThermoFisher Scientific) at −180° C. for data collection. These samples can be similarly examined using a variety of TEMs with cryo-imaging capacity (FIG. 5, step 3).

(2) Cryo-EM Analysis of the N Protein Reveals Unique Structural Features.

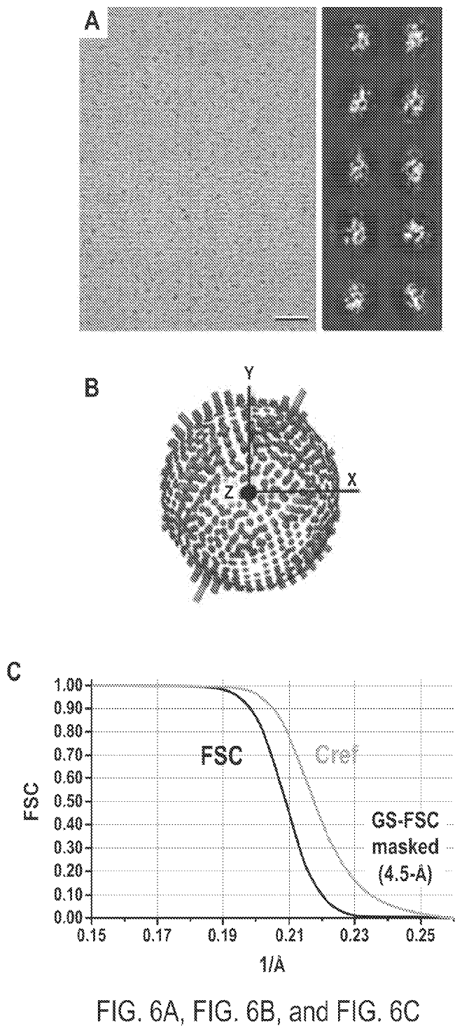

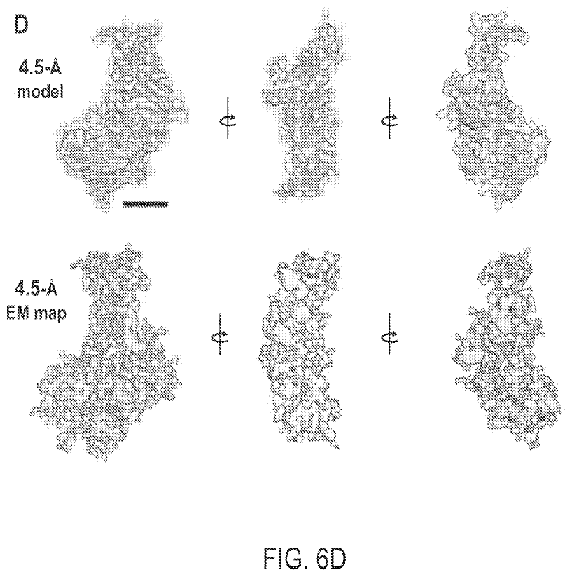

Cryo-EM images were collected under low-dose conditions (<5 electrons/Å²) at 200 kV (FIG. 6A). Movies were acquired at 0.25 second exposures (30 frames per second) with motion-correction at a nominal magnification of ˜45,000×using a DE-12 direct detector. Final sampling at the specimen level was 1.05 Å/pixel. EM structures were computed using standard procedures in the cryoSPARC and RELION software packages (Materials and Methods). The resulting 4.5-Å reconstruction (FIGS. 6B, 6C, and 6D) was interpreted using a model generated with the PHYRE2 software package.

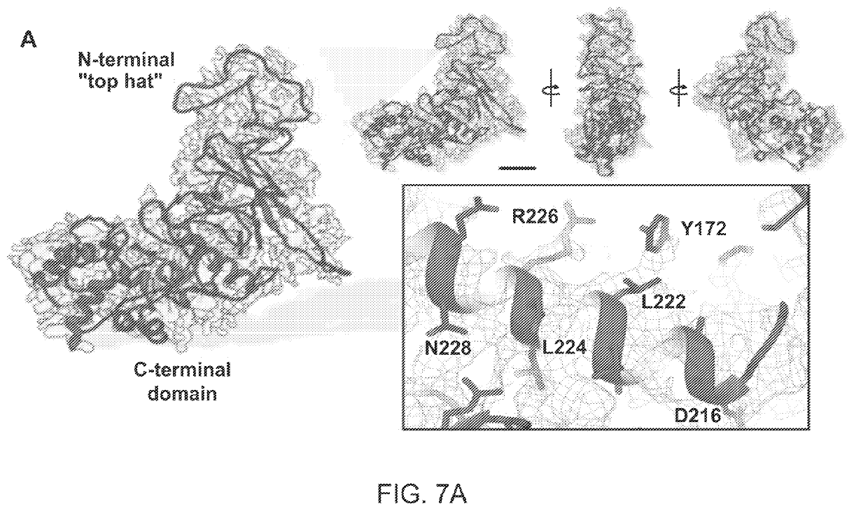

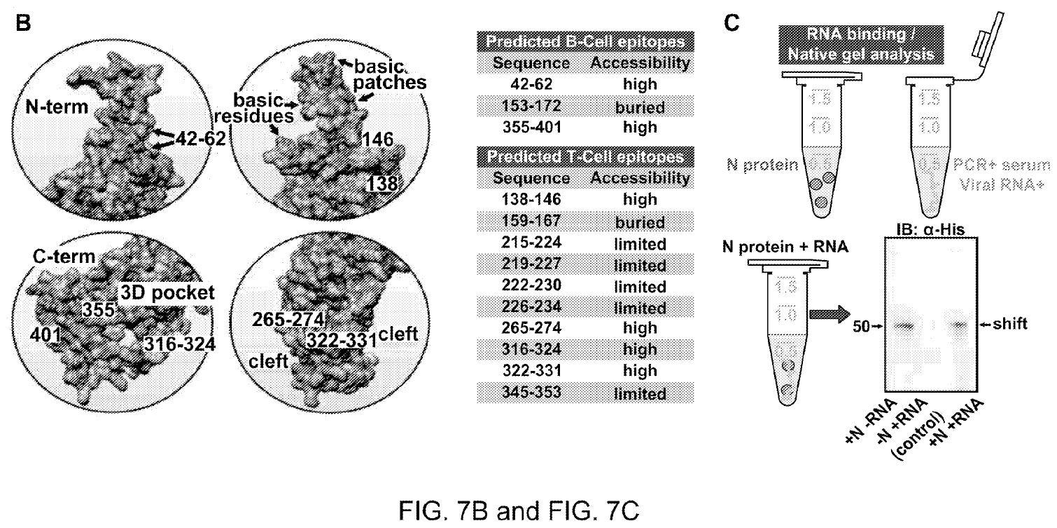

The cryo-EM structure revealed a distinct “top hat” motif in the first 50-amino acids of the N protein (FIG. 7A). According to structure prediction programs, these residues lack secondary elements. We posit that the N-terminal His tag of the recombinant protein served to tether it to the Ni-NTA-coated microchips, providing some level of stability. This action permitted the resolution of this flexible area of the structure without compromising the angular distribution of particle orientations (FIG. 6B). Rotational views and cross-sections through the EM map further support the quality of the model placement within the reconstruction (FIG. 7A). The charge distribution in the N-terminal domain showed both negatively and positively charged areas, along with a basic-rich region containing multiple Arginine residues (FIG. 7B). In comparison to other regions of the protein, the first half of the N-terminus contains the most net positive charge needed to bind to RNA molecules during ribonucleotide-protein interactions and genome packing.

A closer inspection of the central helix found at the boundary between the N- and C-terminal domains reveals density to accommodate the helix defined by residues D216-N228. There is also suitable density for adjacent side chains in this region, although some larger aromatic residues, such as Y172 are not clearly defined. These features are consistent with the expectations at the defined resolution. This helical region is predicted to contain antibody epitopes but is rather inaccessible within the context of the full protein. Another distinguishable part of the structure is the C-terminal region, composed of multiple rigid helices. This area is easily visualized in the lower half of the reconstruction (FIG. 7B). A majority of the residues in C-terminal pockets are either uncharged or hydrophobic residues.

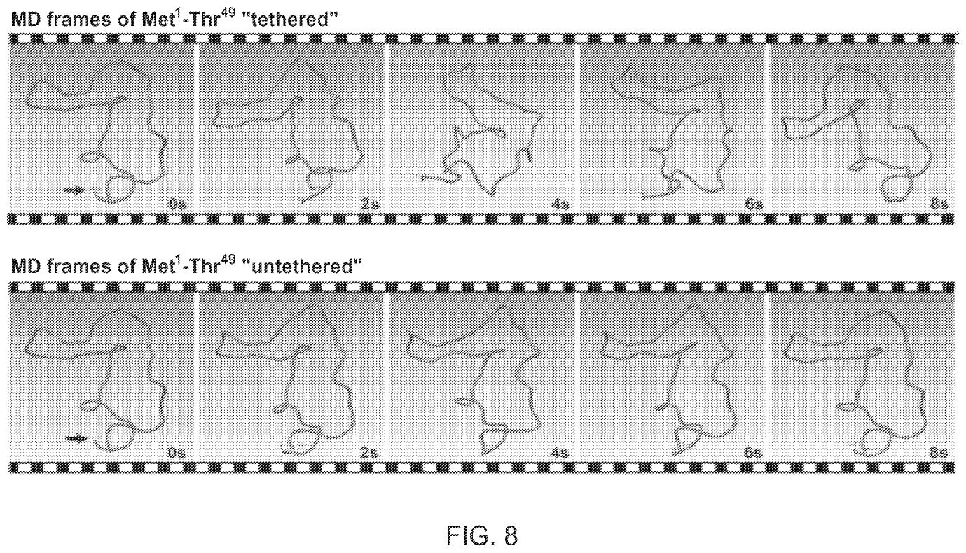

To further understand the impact of the His-tagged tethering process on the 3D structure, molecular dynamics (MD) simulations were performed on the top hat motif using the Chimera software package (FIG. 8). This motif is adjacent to the N-terminal his tag and is most affected by the tethering system. Simulations were conducted with and without holding residue Met¹ anchored in place. This anchoring step represents the attachment of the N-terminal tag to the Ni-NTA coated microchips. Within the sampled timeframe, there were a few rotational differences noted between the two domains.

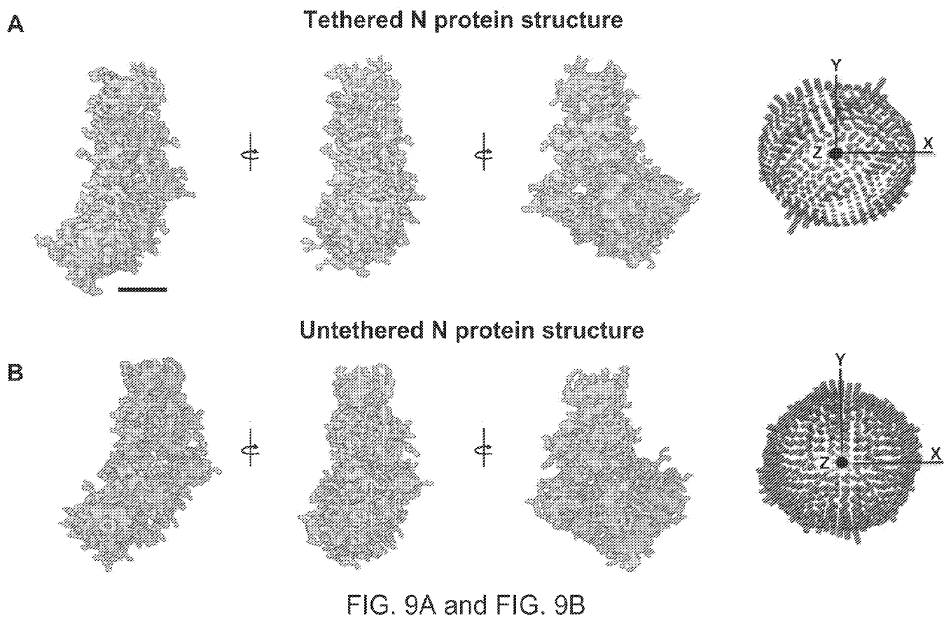

Additional inquiry of how the tagging system influenced the EM structure led to the analysis of untethered protein samples. Reconstructions were calculated using a similar number of particles and the untethered map showed similar features to the tethered structure. The untethered structure may contain more particle views according to angular distribution plots evaluated at the same level of resolution (FIG. 9). These results support a minimal negative impact of the tethering system on the full-length N protein structure.

(3) N Protein Interacts with RNA from PCR+ Patient Serum.

To ensure the functional capacity of the N protein sample, we tested its ability to interact with viral RNA in PCR+ serum from COVID-19 patients (RayBiotech, Inc.) (FIG. 7C). N protein aliquots (0.92 mg/mL in standard PBS buffer) were mixed with varying concentrations of PCR+ serum and incubated at 37° C. for up to 60 minutes. Incubations were halted by the addition of non-reducing gel loading buffer (Invitrogen) and analysed using Western blots of native gels. Primary antibodies against the His-tag were detected in samples containing the N protein. In control samples that lacked viral RNA (RNA−) the N protein migrated on native gels at 50 kDa, consistent with the other analyses. Control samples containing viral RNA (RNA+) but lacked the N protein showed no signal on the Western blot. Samples that were N+/RNA+ showed a shift in the corresponding his-tag signal on the blot. This shift indicates the N protein migrates slower in samples incubated with viral RNA+ serum (FIG. 7C). The gel shift is consistent with protein-RNA interactions typically observed in electrophoretic mobility shift assays. RNA− samples showed no such shift in gel migration. These results support that the His-tagged N protein is capable of associating with RNA substrates in serum samples from COVID-19 patients.

(4) N Protein Binds to Antibodies from COVID-19 Patient Serum.

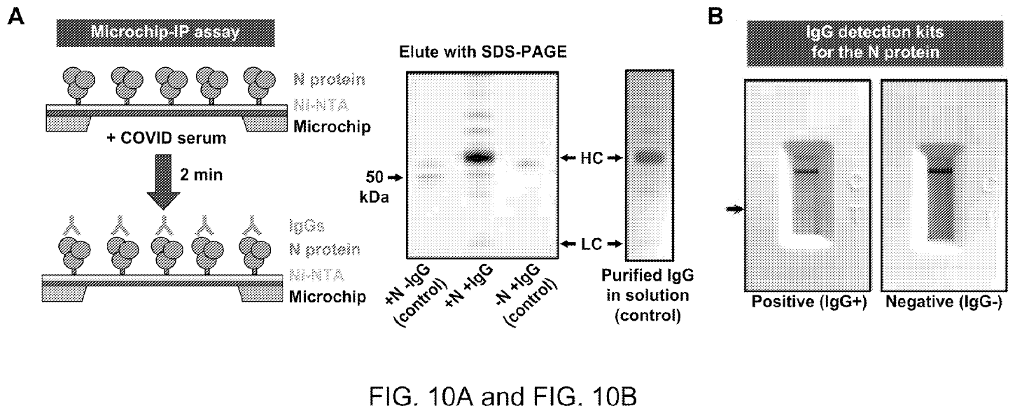

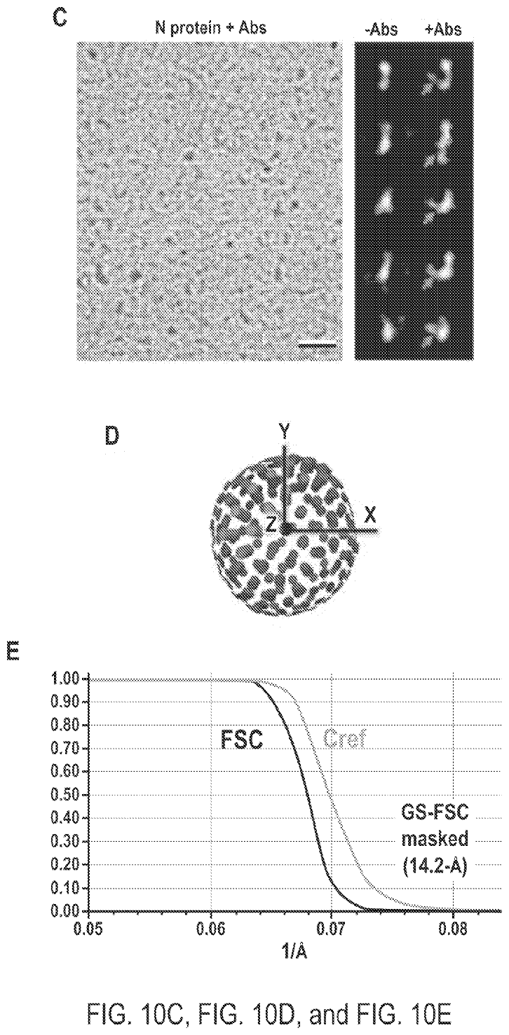

Given the distinct structural elements in the lower-half of the N protein structure, we tested the extent to which these residues formed suitable antibody targets. We first analysed protein sequence data available for B- and T-cell immune responses from patients infected with the related virus, SARS-CoV. Predicted epitopes determined by other research teams were mapped onto the N protein structure and we ranked their level of relative accessibility (FIG. 7B). Predicted B-cell epitopes adjacent to the N-terminal domain or in the C-terminal region showed high accessibility sites and most residues were not buried within the folds of the protein. Predicted T-cell epitopes located in the lower half of the protein, including residues 265-331 were highly accessible, as were residues 138-146. Other predicted sequences within the protein core structure were found to have limited accessibility or were buried. To test for antigenic properties of the N protein, we conducted biochemical and structural experiments using convalescent serum from COVID-19 patients (RayBiotech, Inc.) (FIG. 10).

For antibody binding experiments, we used the same strategy employed in EM preparation steps to develop a rapid microchip-based immunoprecipitation (IP) assay, validated by antibody testing kits (FIG. 10A, 10B). Aliquots of the N protein (0.1 mg/mL in 20 mM Tris (pH 7.5), 150 mM NaCl, 10 mM MgCl₂, 10 mM CaCl₂)) were incubated for one minute with microchips decorated with Ni-NTA coatings. Microchips with and without microwells can be used for these assays. The excess solution was removed, and antibody-containing (IgG+) patient serum (RayBiotech, Inc.) was added to the chips for 2 minutes at room temperature. The excess solution was removed, and the attached proteins were eluted with SDS-based gel loading buffer. For negative controls, we used IgG− patient serum as well as samples that lacked the N protein (N−). Results showed the presence of the N protein migrating at 50 kDa when it was applied to the microchips (N+) (FIG. 10A). For the IgG+/N+ samples, heavy chain (HC) (˜60 kDa) and light chain (LC) (˜25 kDa) components were detected along with the N protein. There was little to no HC or LC signal detected in the IgG− controls or in the N− samples. An additional external control of purified IgGs shows a consistent banding pattern for the antibodies present in the IgG+/N+ samples, demonstrating the N protein specificity for IgGs. The microchip-IP results were reproducible across multiple replicates and microchip samples.

To complement the microchip-IP assay, we tested aliquots of the same patient serum using commercial IgG-specific lateral flow cassettes (RayBiotech, Inc.). These kits contained recombinant his-tagged N protein as the detection analyte. (FIG. 10B). Serum samples were tested in triplicate according to manufactures instructions. Following a 10-minute incubation, the presence of a positive test (T) band on the cassette verified patient-derived IgG antibodies interacted with the N protein analyte (FIG. 10B, black arrow, left). The control (C) band indicates the serum solution is properly migrating through the cassette. Negative control patient serum lacking N protein-specific IgGs did not produce a positive signal in the test region (FIG. 10B, right). These results validated interactions between the N protein and IgGs in the COVID-19 patient serum. Next, we pursued structural studies on the N protein-antibody complexes.

(5) Antibody-Labelling of the N Protein Structure

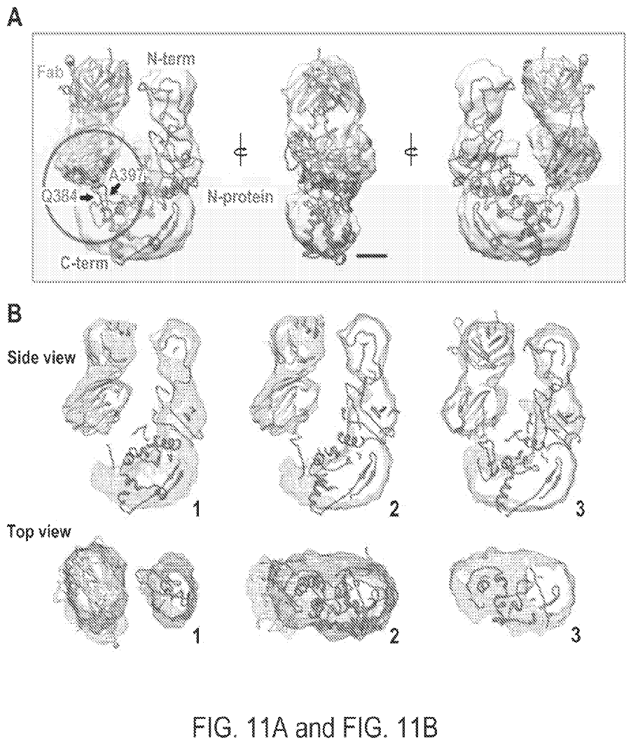

Cryo-EM data was collected on N protein samples decorated with the patient antibodies from parallel biochemical assays. Differences in density were visibly distinct between class averages lacking antibodies (− Abs) and decorated with antibodies (+ Abs). Red arrows point to extra density in the + Abs samples in FIG. 10C. Based on these promising results, we pursued 3D structures of antibody-decorated samples using the same routines implemented for the N protein alone. Particles included in the reconstruction were not limited in their angular orientations and the structure refined to 14.2-Å, according to gold-standard FSC criteria and Cref(0.5) evaluation (FIG. 10D, 10E). Density that accommodates a Fab-sized portion of the antibodies was stably defined in the class averages and EM map. The size and flexible nature of full-length IgG limited the ability to reconstruct the entire molecule. Such results are commonly seen in EM-based antibody-labelling experiments. A closer look at the structure revealed the N protein attachment site for a single Fab domain (FIG. 11). We used a generic model for the Fab structure (pdb code, 4QXG) to interpret the map. The N structure and the Fab model were fit in the reconstruction using the Chimera software package. The Fab binding site on the N protein was located in the C-terminal region at a flexible loop defined by residues Q384-A397 (FIG. 11A).

This loop is highly accessible in the structure and is flanked by two helices that appear to provide stability to the adjacent loop. Rotational views and sections through the EM map demonstrate the quality of the model fit shown in difference views of the structure in FIG. 11B.

Recent crystallographic studies of C-terminal domain constructs reported a putative dimerization site for the N protein structure. The site is located on the opposite side of the newly-defined epitope, such that dimer formation is not be hindered by Fab binding. Additionally, the location of the epitope loop (³⁸⁴QRQKKQQTVTLLPA³⁹⁷) is consistent with predicted sequences based on SARS-CoV comparisons. Bioinformatic studies forecasted a B-cell epitope in residues 355-401. The theoretical mapping of this region showed a highly accessible site, now validated by the structural results. As these tools are a valuable resource to study molecular modelling data, we sought to better understand the relationship of the SARS-CoV-2 N protein to other coronavirus nucleocapsid structures.

(6) Similarities Between Coronavirus Models.

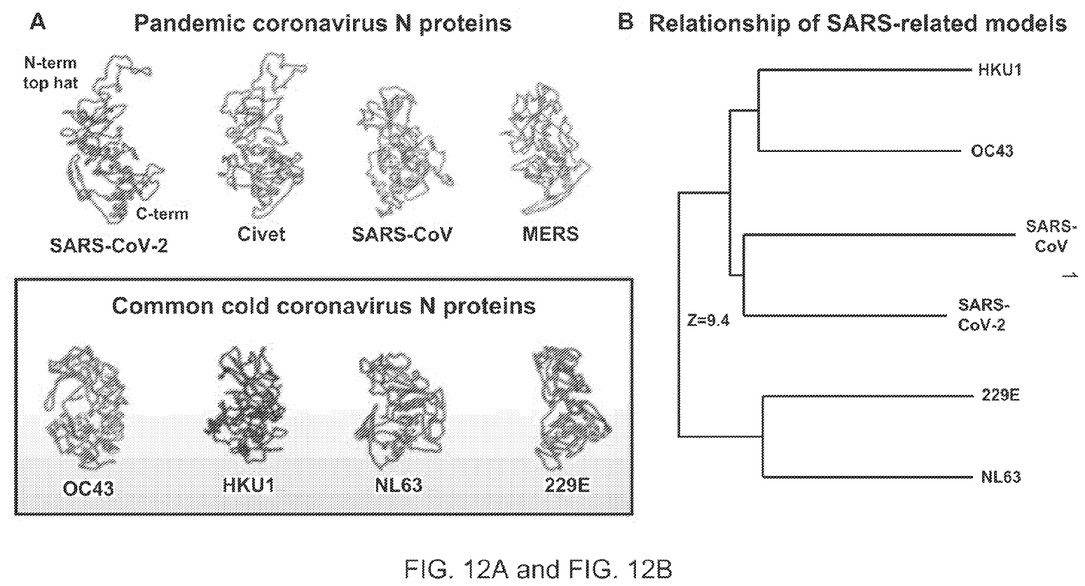

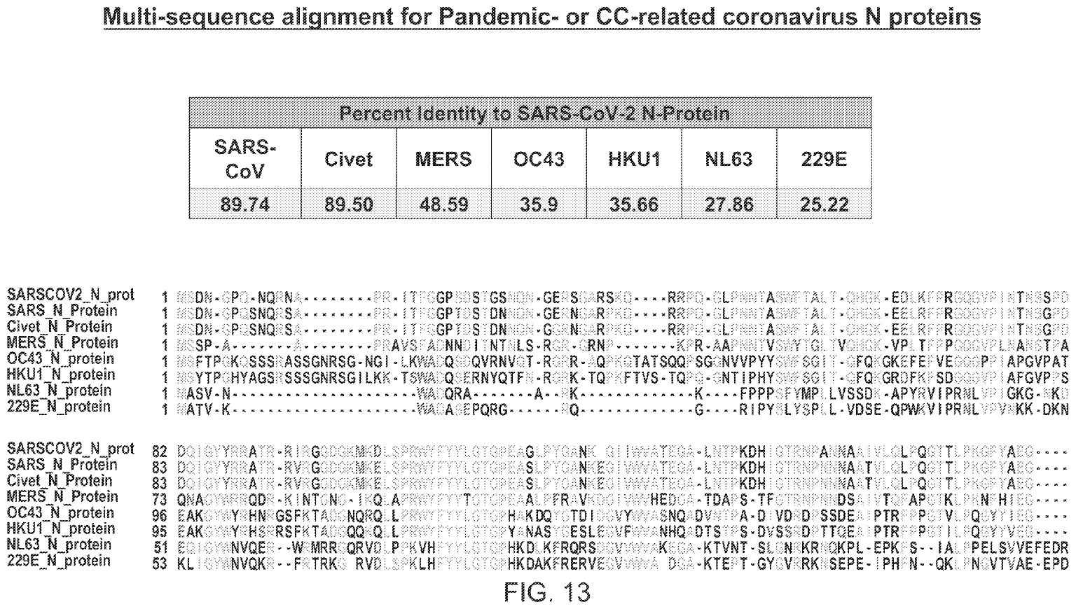

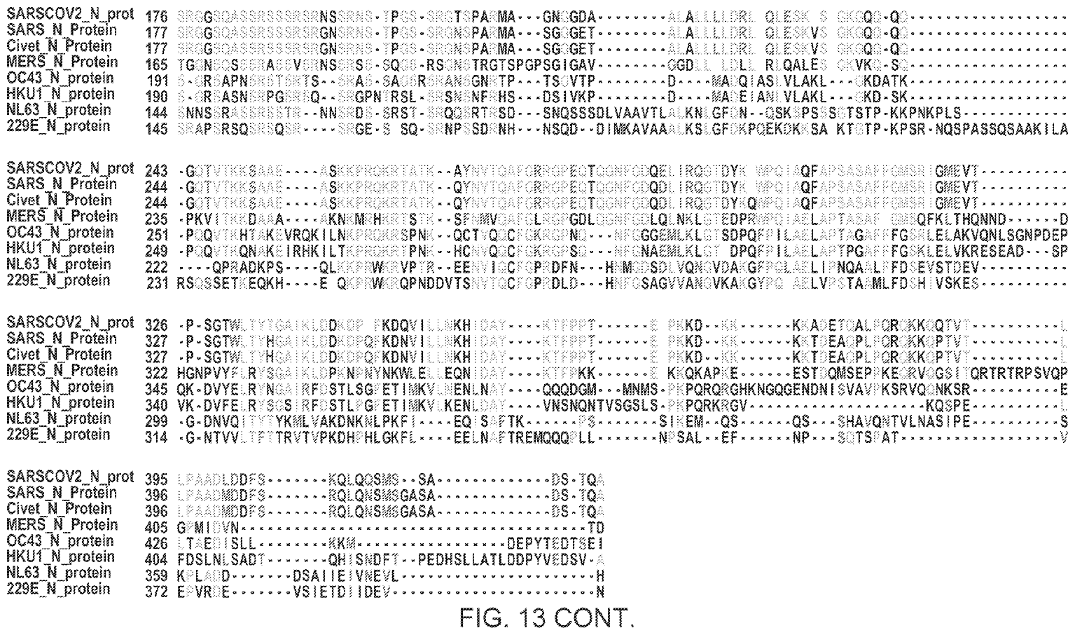

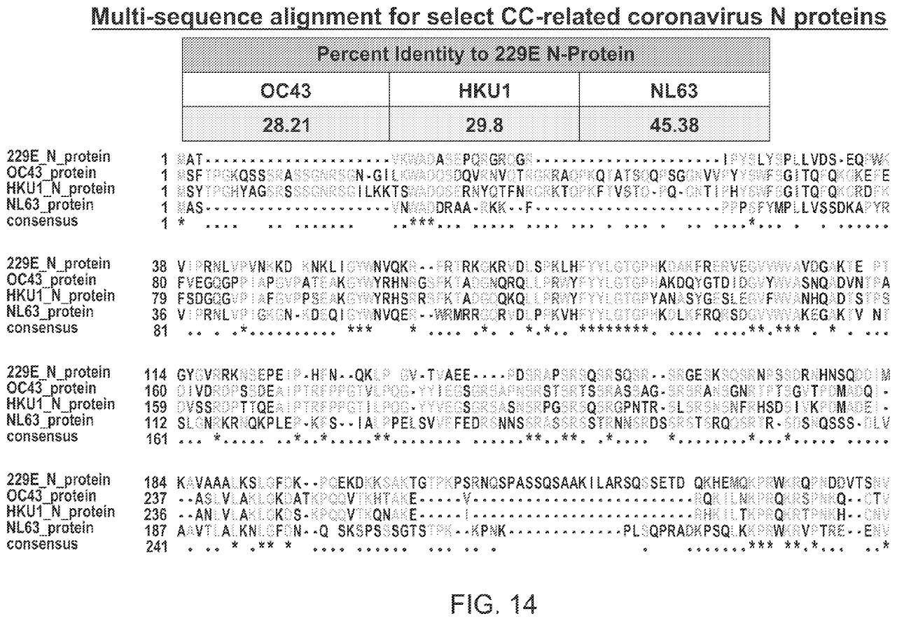

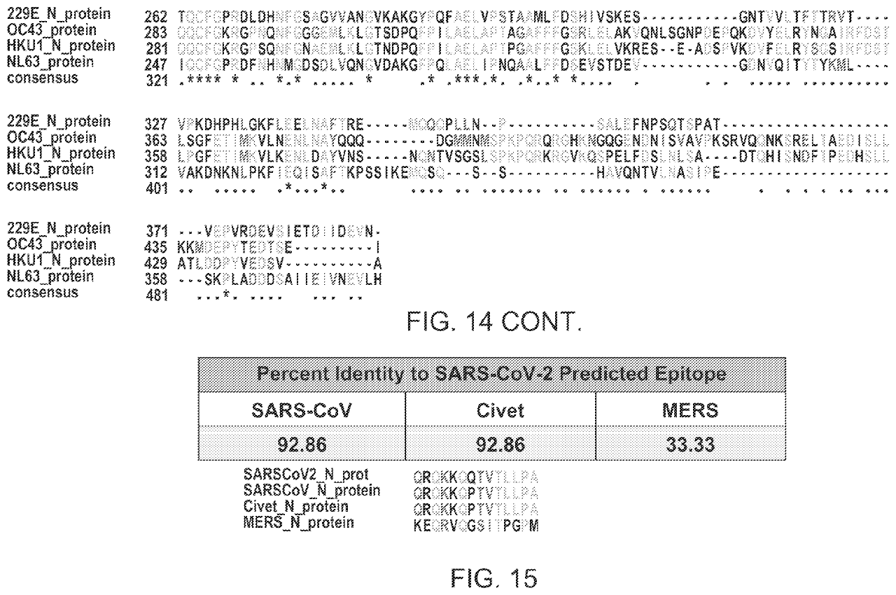

We calculated models for coronavirus strains (HKU1, OC43, 229E, NL63, SARS, MERS) using bioinformatic tools in PHYRE2 (FIG. 12). The common-cold (CC) coronaviruses have lower sequence identities compared with SARS-CoV-2 (<25-36% identity (ID); FIG. 13). However, they do have higher similarities (>28-45% ID; FIG. 14) to CC proteins for which a known crystallographic structure exists (pdb code, 4J3K). Similarities among the CC proteins permitted the production of homology models with a confidence level of >60% with 90% accuracy. These models lacked the N-terminal top-hat motif but showed a helical-rich C-terminal domain (FIG. 12A).

We then examined pandemic-related N protein sequences for MERS (48.6% ID) and SARS-CoV (89.7% ID) along with a coronavirus strain found in Civets (89.5% ID) (Accessed from ViPR database). The Civet model was included in this exercise as it is thought to be an intermediate source for the transition of the original SARS-CoV into humans. Surprisingly, the Civet protein displayed a flexible N-terminal motif similar to SARS-CoV-2, which was lacking in the SARS-CoV and MERS models (FIG. 12A). This unstructured region can convey a unique function during the infection cycle. One idea for its existence is to promote interactions with the viral genome or host RNA. The helical-rich C-terminal domain of pandemic-related proteins was consistent across each model with some variability specific to each structure. Based on the new epitope information for SARS-CoV-2, we posit that distinct loops in the C-terminal domain of these structures can also contain antibody-binding sites. The percent identity in protein sequences for the epitope region (Q384-A397) was SARS—Co-V (93%), Civet (93%), MERS (33%) (FIG. 15 and Table 1).

TABLE 1
Virus sequence information used in our structural analysis

| Virus | GenBank ID |
|---|---|
| SARS-CoV-2 | QJX60119.1 |
| SARS-CoV | AAR86785.1 |
| Civet-CoV | AAU04658.1 |
| MERS | AKK52619.1 |
| OC43 | AXX83383.1 |
| HKUl | ABG77571.1 |
| NL63 | ABI20791.1 |
| 229E | AAA45463.1 |

A list of virus consensus sequences for pandemic and CC coronaviruses is given along with the GenBank ID number.

To graphically illustrate the relationship between models, we quantified structural similarities using the DALI Pairwise Comparison server. Structural comparisons were based on systemic branch and bound searching algorithms. Models for SARS-CoV-2, SARS-CoV, HKU1, OC43, 229E, NL63 were submitted to the server as individual pdb codes. The resulting dendrogram revealed the SARS-CoV-2 and SARS-CoV proteins clustered together branching off from the cluster formed by HKU1 and OC43 (FIG. 12B).

The 229E and NL63 strains were grouped together, branching off further down the tree, reflecting their more distant relationship with the SARS-related models. This result demonstrates appropriate branching sites between alpha- (229E, NL63) and beta-coronaviruses (HKU1, OC43) in comparison to pandemic-related beta-coronaviruses (MERS, SARS-CoV, SARS-CoV-2). Although these branches are somewhat expected, their distributions support the validity of the models while pointing to regions of interest for antibody development.

b) Conclusions

While major advances in the cryo-EM field have focused on instrumentation and hardware technologies, EM substrate development has lagged in comparison. New materials and engineering principles are needed to manufacture highly reproducible samples and to better address difficult questions in structural biology. The microchip-based approach advances the field by integrating engineered substrates for combined structural and biochemical studies of low-molecular weight proteins, 50 kDa or less, which is among the first structures demonstrated in this size range. Samples produced on functionalized substrates provided new information for the SARS-CoV-2 N protein, an important target in COVID-19 therapeutic investigations and rapid testing platforms. Large swaths of useable areas were present on the microchip specimens as previously demonstrated with viral assemblies. Comparatively, samples produced on holey carbon grids or gold foil substrates did not yield specimens suitable for structural analysis. There were no particles present in the imaging holes of these substrates.

New structural results defined a stable 3D epitope in the C-terminal region of the N protein, composed of residues Q384-A397. The antibody-decorated structure was biochemically validated by complementary on-chip and IgG binding assays. On a broader scale, molecular models of various coronavirus proteins demonstrate a visual comparison of immunogenic interaction sites, elevating work by others on protein fold conservation. Remarkably, the data also indicates that many varieties of known coronavirus N proteins have a helical-rich C-terminal domain that likely contains antibody recognition sites. Drawing upon this information, we envision a future roadmap to resolve all proteins of a particular viral pathogenic as outbreaks develop. Efforts focused in this direction may strategically contribute to a pandemic-preparedness war chest of antibody reagents designed to interfere with viral processes. Moreover, the successful application of microchip-based tools to resolve small proteins can open the flood-gate to entities that are difficult to crystallize or are too large for NMR analysis.

Overall, the microchip approach can be used with a multitude of biological products ranging from rotavirus assemblies (˜2 MDa) to the SARS-CoV-2 N protein (˜48 kDa). Ongoing studies are focused on determining higher resolution structures of native proteins with disease relevance. Given the range of opportunity to study large ensembles or small protein constituents, the microchip approach can be a more generalized technique in cryo-EM. While greater degrees of order and stability are always beneficial in structural studies, we now introduce the exciting possibility to resolve intrinsically disordered regions within proteins of interest, once thought impossible.

c) Materials and Methods

(1) Cryo-EM Data Collection and Image Processing.

Images were recorded under low-dose conditions (<5 electrons/Å²/s) at 200 kV. Sixty movies were acquired at 0.25 second exposures (30 frames per second) with motion-correction at a nominal magnification of ˜45,000×using a DE-12 direct detector. Final sampling at the specimen level was 1.05 Å/pixel. EM structures were calculated using standard procedures in the cryoSPARC software package. Briefly, movies were processed in cryoSPARC with CTF parameters estimated using CTFFIND-4.1 integrated into the package. The spherical aberrations, voltage, amplitude contrast values were set to 2.7 mm, 200 kV, 0.1 respectively for CTF estimation. Particles were manually picked and extracted with a box size of 200 pixels. Extracted particles were classified and sub-selected as templates for auto-picking including ˜20,000 particles. A low-resolution model based on EM data was initially calculated in the RELION software package using ab initio methods and C1 symmetry. The low-pass filtered initial model was imported into cryoSPARC and particles were subjected to standard 3D classification and refinement procedures. The final density map and corresponding structural resolution were validated alongside parallel processing procedures in RELION using gold-standard methods (0.143-FSC criteria, Cref (0.5)) in cryoSPARC, RELION, and the RMEASURE executable. The same computational routines were implemented for the untethered N protein structure as wells as the structure containing Fab antibody fragments. The final N-Fab structure was 14.2-Å and contained ˜10,000 particles. Resolution was validated using gold-standard methods (0.143-FSC criteria, Cref(0.5)) in cryoSPARC and the RMEASURE executable.

(2) Structure Prediction and Bioinformatics.

N-protein structure prediction was performed by uploading the primary sequences of each of the designated N-proteins to the PHYRE2 protein fold recognition server. The viral protein sequences were obtained from the Virus Pathogen Database and Analysis Resource (ViPR). Graphical representations and spatial alignments were performed using UCSF Chimera, and movies illustrating the relationship between proteins were created using the Blender software package. Primary amino acid sequences obtained from ViPR were uploaded to the ClustalOmega online software package to generate percent identity matrices. Amino acid sequences were then uploaded to the T-Coffee MSA server to generate multiple sequence alignments (FIG. 13, 14, 15). The resulting file was then uploaded to the BoxShade server to aid in visualization of results.

(3) Molecular Modeling Analysis.

PDB files of homology models generated using PHYRE2 were uploaded to the Dali server's Pairwise structure comparison pipeline. Structural dendrograms were produced by the server grouping proteins based on the calculated similarity matrix (Z-score). These groupings show which proteins have similar features. The resulting dendrogram was reformatted in Adobe Photoshop to facilitate ease of reading and manuscript formatting.

(4) N Protein Validation.

Purified N-protein (RayBiotech, 230-01104-100) was analyzed using denaturing (SDS-PAGE) or Native gels to confirm homogeneity. Gels were stained in SimplyBlue SafeStain overnight according and imaged using a BioRad ChemiDoc MP. The N protein (100 ng) was also analyzed via immunoblotting in parallel with SimplyBlue analysis to further confirm integrity of N-protein samples. Following electrophoresis, proteins were transferred to a PVDF membrane at 80 V in 1×NUPAGE transfer buffer for 60 minutes. The membranes were incubated in TBS-T supplemented with 3% BSA for 16 hours with gentle agitation. After blocking, membranes were incubated with antibodies raised against a 6×-His tag (GenScript, A00186) in TBS-T supplemented with 3% BSA at a dilution of 1:1000 at 25° C. for 60 minutes with gentle agitation. Following primary antibody incubation, membranes were washed with TBS-T with vigorous agitation. Membranes were incubated with α-mouse IgG-HRP conjugated secondary antibodies (1:10,000) in TBS-T for 60 minutes with gentle agitation. BioRad Clarity Max ECL blotting solution was used according to manufacturer's recommendations before visualization with a BioRad ChemiDoc MP.

(5) RNA Shift Assays.

500 ng of His-tagged N-protein (0.92 mg/mL, RayBiotech, 230-01104-100) were incubated with 2.5 μg SARS-CoV-2 PCR+(105 mg/mL; RayBiotech, CoV-PosPCR-S-100) human serum or SARS-CoV-2 PCR− human serum (103 mg/mL; RayBiotech, CoV-NegPCR-S-100) where indicated. Reactions were brought to a final volume of 20 μL with PBS. Reaction mixtures were then incubated at 25° C. or 37° C. for 60 minutes. After the indicated reaction time, sample loading buffer containing no SDS was added to each reaction mixture and samples were loaded into a NuPage Bis-Tris 4-12% polyacrylamide gel and allowed to migrate at 180 V for 60 minutes. After electrophoresis, proteins were transferred to a PVDF membrane using lx NuPage transfer buffer at 80 V for 60 minutes. PVDF membranes were then incubated in TBS-T supplemented with 3% BSA at 4° C. with gentle agitation for 16 hours. Membranes were incubated with a primary antibody against 6×-His tag (GenScript, A00186) in TBS-T supplemented with 3% BSA at 25° C. for 60 minutes with gentle agitation. Membranes were washed with TBS-T and agitation then incubated with an α-mouse IgG HRP-conjugated secondary antibody in TBS-T supplemented with 3% BSA for 60 minutes. Following secondary antibody incubation, membranes were washed with TBS-T for 10 minutes. Membranes were incubated with BioRad Clarity Max ECL substrate (1705062) according to manufacturer recommendations for visualization using a BioRad ChemiDoc MP.

(6) Antibody Binding Assays.

Aliquots (5 μL) of His-tagged N protein (0.1 mg/mL in 20 mM Tris (pH 7.5), 150 mM NaCl, 10 mM MgCl2, 10 mM CaCl₂)) were added to Ni-NTA-coated microchips (Protochips, Inc, EPT-52W) and incubated for 1 minute at room temperature. Control samples were incubated with Tris buffer solution that lacked the N protein. The excess solution was blotted away with Whatmann filter paper followed by the addition of either IgG+(CoV-PosG-S-100) or IgG− (CoV-NegG-S-100) COVID-19 serum samples (0.3 mg/mL in 20 mM Tris (pH 7.5), 150 mM NaCl, 10 mM MgCl2, 10 mM CaCl2)). Samples were incubated for 2 minutes at room temperature after which time the excess solution was blotted away with Whatmann filter paper. Chip contents were eluted with SDS-PAGE buffer solution (˜10 μl per chip) and samples were assessed using 4-12% polyacrylamide gels and standard electrophoresis protocols. Gels were stained using SimplyBlue and visualized with a BioRad ChemiDoc MP.

(7) Antibody Detection Kits.

COVID-19 IgG rapid test kit cassettes (CG-CoV-IgG-RUO) were purchased from RayBiotech, Inc. along with IgG+(CoV-PosG-S-100) and IgG− (CoV-NegG-S-100) serum samples. Aliquots of serum (25 μL) were mixed with the supplied sample buffer and applied to the sample area of the kit. Tests were read within 10 minutes of sample application. Three tests were run for each IgG+ or IgG− samples and no false positives were detected.

### E. SEQUENCES

```
amino acid sequence of SARS-CoV-2 N-protein
SEQ ID NO: 1
                10 20 30 40
MSDNGPQNQR NAPRITFGGP SDSTGSNQNG ERSGARSKQR
                50 60 70 80
RPQGLPNNTA SWFTALTQHG KEDLKFPRGQ GVPINTNSSP
                90 100 110 120
DDQIGYYRRA TRRIRGGDGK MKDLSPRWYF YYLGTGPEAG
              130 140 150 160
LPYGANKDGI IWVATEGALN TPKDHIGTRN PANNAAIVLQ
              170 180 190 200
LPQGTTLPKG FYAEGSRGGS QASSRSSSRS RNSSRNSTPG
              210 220 230 240
SSRGTSPARM AGNGGDAALA LLLLDRLNQL ESKMSGKGQQ
              250 260 270 280
QQGQTVTKKS AAEASKKPRQ KRTATKAYNV TQAFGRRGPE
              290 300 310 320
QTQGNFGDQE LIRQGTDYKH WPQIAQFAPS ASAFFGMSRI
              330 340 350 360
GMEVTPSGTW LTYTGAIKLD DKDPNFKDQV ILLNKHIDAY
              370 380 390 400
KTFPPTEPKK DKKKKADETQ ALPQRQKKQQ TVTLLPAADL
              410
DDFSKQLQQS MSSADSTQA
amino acid sequence B cell epitope binding motif
SEQ ID NO: 2
X¹RQX²KQX³X⁴X⁵TLLPA; wherein X¹ is Q or K; wherein
X² is K or R; wherein X³ is P. Q, or G; wherein
X⁴ is T or S; and wherein X⁵ is V or I
amino acid residues 384-397 of SARs-CoV2
SEQ ID NO: 3
QRQKKQQTVTLLPA
```

What is claimed is:

1. A method of screening for an anti-coronavirus therapeutic agent comprising contacting a coronavirus N protein with a therapeutic agent; wherein a therapeutic agent that binds to the N protein N-terminal “top hat” motif (residues 1-62 as set forth in SEQ ID NO: 1) or the N protein C-terminal helix (residues 362-419 as set forth in SEQ ID NO: 1) is an anti-coronavirus therapeutic agent.

2. The method of claim 1, wherein the interaction of the therapeutic agent and the N protein is measured by cryoEM.

3. The method of claim 1 or 2, wherein the therapeutic agent binds a B cell epitope selected from the group consisting of residues 42-62 or residues 355-401, as set forth in SEQ ID NO: 1.

4. The method of claim 1 or 2, wherein the therapeutic agent binds the sequence motif X¹RQX²KQX³X⁴X⁵TLLPA; wherein X¹ is Q or K; wherein X² is K or R; wherein X³ is P, Q, or G; wherein X⁴ is T or S; and wherein X⁵ is V or I.

5. The method of claim 3 or 4, wherein the therapeutic agent the amino acid sequence as set forth in SEQ ID NO: 3.

6. The method of claim 1 or 2, wherein the therapeutic agent binds a T cell epitope selected from the group consisting of residues 138-146, residues 159-167, residues 215-224, residues 219-227, residues 222-230, residues 226-234, residues 265-274, residues 316-324, residues 322-331, or residues 345-353 as set forth in SEQ ID NO: 1.

7. An anti-coronavirus therapeutic agent identified by the method of any of claims 1-6.

8. An anti-coronavirus therapeutic agent; wherein the anti-coronavirus therapeutic agent binds to the N-terminal “top hat” motif (residues 1-62) or the C-terminal helix (residues 362-419) of the nucleocapsid (N) protein of a coronavirus.

9. The anti-coronavirus therapeutic agent of claim 8, wherein the therapeutic agent binds a B cell epitope selected from the group consisting of residues 42-62 or residues 355-401 as set forth in SEQ ID NO: 1.

10. The anti-coronavirus therapeutic agent of claim 8 or 9, wherein the therapeutic agent binds the sequence motif X¹RQX²KQX³X⁴X⁵TLLPA; wherein X¹ is Q or K; wherein X² is K or R; wherein X³ is P, Q, or G; wherein X⁴ is T or S; and wherein X⁵ is V or I.

11. The anti-coronavirus therapeutic agent of claim 10, wherein the amino acid sequence as set forth in SEQ ID NO: 3.

12. The anti-coronavirus therapeutic agent of claim 8, wherein the therapeutic agent binds a T cell epitope or B cell epitope selected from the group consisting of residues 42-62, residues 153-172, residues 355-401, residues 138-146, residues 159-167, residues 215-224, residues 219-227, residues 222-230, residues 226-234, residues 265-274, residues 316-324, residues 322-331, or residues 345-353 as set forth in SEQ ID NO: 1.

13. The anti-coronavirus therapeutic agent of any of claims 7-12, wherein the therapeutic agent comprises a small molecule, antibody, antibody fragment, RNAi, siRNA, peptide, or protein, or any combination thereof.

14. A recombinant virus or virus-like particle (VLP) comprising nucleic acid encoding the N protein of a coronavirus or a fragment thereof.

15. The recombinant virus or VLP of claim 14, wherein the recombinant virus or VLP comprises a nucleic acid encoding the amino acid sequence motif X¹RQX²KQX³X⁴X⁵TLLPA; wherein X¹ is Q or K; wherein X² is K or R; wherein X³ is P, Q, or G; wherein X⁴ is T or S; and wherein X⁵ is V or I.

16. The recombinant virus or VLP of claim 14 or 15, wherein the N-protein fragment comprises the N-terminal “top hat” motif of the N-protein and/or the C-terminal helix of the N-protein.

17. The recombinant virus or VLP of any of claims 14-16, wherein the recombinant virus or VLP comprises a nucleic acid encoding the amino acids as set forth in SEQ ID NO: 3.

18. The recombinant virus or VLP of any of claims 14-16, wherein the recombinant virus or VLP comprises a nucleic acid encoding amino acid residues 138-146, residues 159-167, residues 215-224, residues 219-227, residues 222-230, residues 226-234, residues 265-274, residues 316-324, residues 322-331, or residues 345-353 as set forth in SEQ ID NO: 1.

19. The recombinant virus or VLP of any of claims 14-18, wherein the N protein of a coronavirus or a fragment thereof is derived from the N-protein of a coronavirus selected from the group consisting of avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2, and middle east respiratory syndrome (MERS) coronavirus (CoV) (MERS-CoV).

20. An immunogenic composition comprising one or any combination of two or more of a N-terminal “top hat” motif or the C-terminal helix of the nucleocapsid (N) protein of a coronavirus, virus comprising the N protein of a coronavirus or a fragment thereof, virus like particle, or an antibody or antibody fragment to the N-terminal “top hat” motif or the C-terminal helix of the N protein of a coronavirus.

21. The immunogenic composition of claim 20, wherein composition comprises the sequence motif X¹RQX²KQX³X⁴X⁵TLLPA; wherein X¹ is Q or K; wherein X² is K or R; wherein X³ is P, Q, or G; wherein X⁴ is T or S; and wherein X⁵ is V or I

22. The immunogenic composition of claim 20 or 21, wherein composition comprises residues 138-146, residues 159-167, residues 215-224, residues 219-227, residues 222-230, residues 226-234, residues 265-274, residues 316-324, residues 322-331, or residues 345-353 as set forth in SEQ ID NO: 1.

23. A method of treating a subject with a coronavirus comprising administering to the subject the anti-coronavirus therapeutic agent of any of claims 7-13, recombinant coronavirus or virus-like particles (VLPs) of any of claims 14-19, or the immunogenic composition of any of claims 20-22.

24. The method treating a subject with a coronavirus of claim 23, wherein the coronavirus comprises avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2), and middle east respiratory syndrome (MERS) coronavirus (CoV) (MERS-CoV).

25. A method of preventing or inhibiting a coronavirus infection in a subject comprising administering to the subject the anti-coronavirus therapeutic agent of any of claims 7-13, recombinant coronavirus or virus-like particles (VLPs) of any of claims 14-19, or the immunogenic composition of any of claims 20-22.

26. The method preventing or inhibiting a coronavirus infection of claim 25, wherein the coronavirus comprises avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2), and middle east respiratory syndrome (MERS) coronavirus (CoV) (MERS-CoV).

27. A method of preventing or inhibiting a Severe Acute Respiratory Syndrome (SARS)-Coronavirus (CoV)-2 (SARS-CoV-2) infection in a subject comprising immunizing the subject with human coronavirus (HCoV)-229E, HCoV-OC43, HCoV-HKU1, HCoV-NL63 or a recombinant virus or virus-like particle (VLP) comprising the N protein of a coronavirus or a fragment thereof.

28. The method of preventing or inhibiting a SARS-CoV-2 of claim 27, wherein the N-protein fragment comprises the N-terminal “top hat” motif of the N-protein and/or the C-terminal helix of the N-protein.

29. The method of preventing or inhibiting a SARS-CoV-2 of claim 27 or 28, wherein the N-protein fragment comprises the sequence motif X¹RQX²KQX³X⁴X⁵TLLPA; wherein X¹ is Q or K; wherein X² is K or R; wherein X³ is P, Q, or G; wherein X⁴ is T or S; and wherein X⁵ is V or I.

30. The method of preventing or inhibiting a SARS-CoV-2 of claim 27 or 28, wherein the N-protein fragment comprises the amino acid sequence as set forth in SEQ ID NO: 3.

31. The method of preventing or inhibiting a SARS-CoV-2 of claim 27 or 28, wherein the N-protein fragment comprises residues 138-146, residues 159-167, residues 215-224, residues 219-227, residues 222-230, residues 226-234, residues 265-274, residues 316-324, residues 322-331, or residues 345-353 as set forth in SEQ ID NO: 1.

32. The method of preventing or inhibiting a SARS-CoV-2 of any of claims 27-31, wherein the N protein of a coronavirus or a fragment thereof is derived from the N-protein of a coronavirus selected from the group consisting of avian coronavirus (IBV), porcine coronavirus HKU15 (PorCoV HKU15), Porcine epidemic diarrhea virus (PEDV), human coronavirus (HCoV)-229E, HCoV-OC43, HCoV-HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2, and middle east respiratory syndrome (MERS) coronavirus (CoV) (MERS-CoV).
